(19) United States
(12) Patent Application Publication
(10) Pub. No.: US 2020/0368166 A1
(43) Pub. Date: Nov. 26, 2020

(54) Oral Formulations And Uses Thereof

(71) Applicant: Eustralis Pharmaceuticals Limited (Trading As Pressura Neuro), Melbourne, Victoria (AU)

(72) Inventors: Pierre Vankan, Melbourne, Victoria (AU); Andreea Sasarman, Melbourne, Victoria (AU); Grasiela Bourscheit Willmbrink, Melbourne, Victoria (AU)

(73) Assignee: Eustralis Pharmaceuticals Limited (Trading As Pressura Neuro), Melbourne, Victoria (AU)

(21) Appl. No.: 16/966,289

(22) PCT Filed: Feb. 1, 2019
(86) PCT No.: PCT/AU2019/050076

§ 371 Date: Jul. 30, 2020

(30) Foreign Application Priority Data
Feb. 2, 2018 (AU) 2018900324

(51) Int. Cl. A61K 9/20; A61K 31/496; A61K 9/16; A61K 9/50

(52) U.S. Cl. CPC A61K 9/2081; A61K 31/496; A61K 9/1623; A61K 9/1617; A61K 9/1641; A61K 9/1652; A61K 9/2059; A61K 9/5047; A61K 9/2009; A61K 9/2013; A61K 9/2018; A61K 9/2054; A61K 9/1611

(57) ABSTRACT

Disclosed are therapeutic oral formulations comprising particular substituted pyridine based compounds, their manufacture, and methods and uses of the formulations in treating substance P mediated pathways in the brain such as elevated intracranial pressure or the modification of expression of (hyper)-phosphorylated tau protein (τ) in the brain for indications such as, but not limited to concussion, post-concussive (or post-concussion) syndrome (PCS), chronic traumatic encephalopathy (CTE), traumatic brain injury (TBI) and stroke.

### FIELD

This invention relates generally to therapeutic oral formulations comprising particular substituted pyridine based compounds, their manufacture, and methods and uses of said formulations in treating substance P mediated pathways in the brain such as elevated intracranial pressure or the modification of expression of (hyper)-phosphorylated tau protein (τ) in the brain for indications such as, but not limited to concussion, post-concussive (or post-concussion) syndrome (PCS), chronic traumatic encephalopathy (CTE), traumatic brain injury (TBI) and stroke.

### BACKGROUND

Traumatic brain injury (TBI), also known as intracranial injury, occurs when an external force injures the brain. TBI can be classified based on severity, mechanism (closed or penetrating head injury), or other features (e.g., occurring in a specific location or over a widespread area). TBI can result in physical, cognitive, social, emotional, and behavioural symptoms, and outcomes can range from complete recovery to permanent disability or death.

Brain trauma occurs as a consequence of a sudden acceleration or deceleration within the cranium or by a complex combination of both movement and sudden impact. In addition to the damage caused at the moment of injury, a variety of events in the minutes to days following the injury may result in secondary injury. These processes include alterations in cerebral blood flow and the pressure within the skull as well as expression of (hyper)-phosphorylated tau protein (τ) in the brain,

The most common causes of TBI include violence, transportation accidents, construction, and sports. Motor bikes are major causes, increasing in significance in developing countries as other causes reduce. It is estimated that between 1.6 and 3.8 million traumatic brain injuries each year are a result of sports and recreation activities in the US. In children aged two to four, falls are the most common cause of TBI, while in older children traffic accidents compete with falls for this position. TBI is the third most common injury to result from child abuse. Abuse causes 19% of cases of paediatric brain trauma, and the death rate is higher among these cases.

There is a lack of effective medication that can lower elevated intracranial pressure (ICP) in TBI or stroke, neither is there any medication that can prevent the over-expression of hyper-phosphorylated tau protein which has been linked to bad clinical outcome in indications such as TBI but also Alzheimer's disease. Accordingly, there exists a need for a medication that can cure or ameliorate elevated ICP in TBI or stroke or prevent over-expression of hyper-phosphorylated tau protein.

Even while an active pharmaceutical ingredient (API) is identified, there are still many obstacles to overcome in formulating a drug. In formulating a drug suitable for human administration, the skilled person would be aware that the formulation art is not predictable. Various factors need to be carefully investigated and tuned to at least maintain (if not enhance) the pharmacokinetic properties of the API, and/or impart stability to the drug such that it can have an acceptable shelf-life. In this sense, the physical characteristic of the API, the mode of delivery, the flow properties of the composition, the excipient compatibility, the uniformity in production and the release profile needs to be carefully studied and investigated.

If not properly formulated, the API may not efficiently provide bioavailability to a patient. For example, while calcium salts can be utilized as fillers, it was found that they also interfere with the absorption of tetracycline (an example of an API) from the gastrointestinal tract. This one example emphasizes that components added in formulations may not always be inert, as one may perceive, and can interact with the API.

Further, the addition of diluents into a formulation may also alter the physical-chemical properties of the formulation which may render the product unstable and may cause problems in manufacturing. This is further compounded by the need for Good Manufacturing Practice (GMP) standards, as certain compliance of each ingredient with existing standards and regulations must be met in a pharmaceutical formulation for use as a drug.

The present invention seeks to overcome or ameliorate at least one of the shortcomings of the art in respect to the formulation of specific compounds.

### SUMMARY OF THE INVENTION

The present invention provides a therapeutic oral formulation that comprises an effective amount of a particular substituted pyridine based compounds and other excipients, and optionally a coating. In particular, the formulation is provided in the form of a tablet. The tablet is characterised by a consistent weight and content uniformity, good dissolution profile and acceptable hardness. Accordingly, the tablet is able to achieve an immediate release dissolution profile. In this regard, the formulation would be able to benefit a subject in need thereof by providing instant relief of substance P mediated processes such as over-expression of hyper-phosphorylated tau protein or elevated intracranial pressure (ICP) and accordingly immediately alleviate the condition and/or symptom of indications as such, but not limited to PCS, CTE, TBI and stroke.

In a first aspect, the present invention provides a pharmaceutical composition in the form of a tablet comprising:
(i) a compound of Formula (I) or a pharmaceutically acceptable salt, solvate or prodrug thereof:

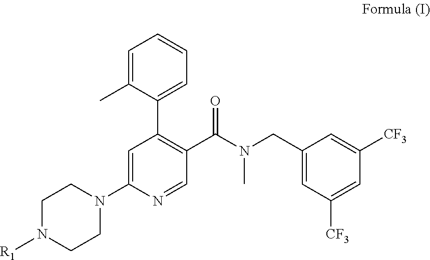

wherein R₁ is H or C₁₋₄ alkyl; and
wherein the compound of Formula (I) or the pharmaceutically acceptable salt, solvate or prodrug thereof within the composition has a D(0.5) particle size distribution of less than about 60 μm;
(ii) at least one diluent selected from the group consisting of lactose, sorbitol, dibasic calcium phosphate dihydrate, calcium sulphate dihydrate, calcium carbonate, croscarmellose sodium, calcium phosphate, calcium hydrogen phosphate dihydrate, crospovidone, ferric oxide, magnesium carbonate, magnesium oxide, sucrose, or sodium chloride, wherein the at least one diluent is present in the composition in an amount from about 35% to about 70% wt/wt based on the total weight of the composition;
(iii) at least one lubricant selected from the group consisting of magnesium stearate, stearic acid, calcium stearate, paraffin, sodium lauryl sulphate, sodium benzoate, castor oil hydrogenated, glyceryl monostearate, glyceryl behenate, sodium stearyl fumarate, mineral oil, polaxamer, PEG 400, PEG 600, or PEG 8000, wherein the at least one lubricant is present in the composition in an amount from about 0.1% to about 2% wt/wt based on the total weight of the composition;
(iv) at least one disintegrant selected from the group consisting of microcrystalline cellulose, alginic acid, citric acid, croscamellose sodium, carboxy methyl cellulose calcium, cysteine HCl, methyl cellulose, polyoxy stearate, sodium starch glycolate, sodium alginate, or carboxy methyl cellulose sodium, wherein the at least one disintegrant is present in the composition in an amount from about 20% to about 30% wt/wt based on the total weight of the composition;
(v) at least one binder selected from the group consisting of starch, gelatin, glucose, polyvinyl pyrrolidone (Povidone), carboxymethylcellulose, acacia, candelilla wax, carnuba wax, cornstarch, glyceryl behenate, hypromellose, or polyethylene oxide, wherein the at least one binder is present in the composition in an amount from about 5% to about 15% wt/wt based on the total weight of the composition; and
(vi) at least one anti-caking agent selected from the group consisting of fumed silica, silicon dioxide, or talc, wherein the at least one anti-caking agent is present in the composition in an amount from about 0.2% to about 2% wt/wt based on the total weight of the composition.

In an embodiment, the present invention provides a pharmaceutical composition as described herein, wherein the compound of Formula (I) or the pharmaceutically acceptable salt, solvate or prodrug thereof is present in the composition in an amount from about 0.1% to about 50% wt/wt based on the total weight of the composition.

In a second aspect, the present invention provides a method for preventing the over-expression of hyperphosphorylated tau protein after a concussion, in a subject in need thereof, the method comprising administering to the subject a pharmaceutical composition in the form of a tablet as described herein.

In a third aspect, the present invention provides a pharmaceutical composition in the form of a tablet for use in preventing the over-expression of hyperphosphorylated tau protein after a concussion, in a subject in need thereof, the pharmaceutical composition is as described herein.

In a fourth aspect, the present invention provides a method for treating elevated intracranial pressure in a subject in need thereof, the method comprising administering to the subject a pharmaceutical composition in the form of a tablet as described herein.

In a fifth aspect, the present invention provides a pharmaceutical composition in the form of a tablet for use in the treatment of elevated intracranial pressure in a subject in need thereof, the pharmaceutical composition is as described herein.

In an embodiment, the method for preventing the over-expression of hyperphosphorylated tau protein is a method for treating concussion or Post-Concussion Syndrome (PCS).

In an embodiment, the method for treating elevated intracranial pressure is a method for treating traumatic brain injury.

In another embodiment, the method for treating elevated intracranial pressure is a method for treating stroke.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “about” or “approximately” as used herein means within an acceptable error range for the particular value as determined by one of ordinary skill in the art, which will depend in part on how the value is measured or determined, i.e., the limitations of the measurement system.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Unless defined otherwise, all technical and scientific terms used herein have the same meaning as commonly understood by those of ordinary skill in the art to which the invention belongs. For the purposes of the present invention, the following terms are defined below.

“Alkyl” refers to monovalent alkyl groups which may be straight chained or branched and have from 1 to 4 carbon atoms or more preferably 1 to 3 carbon atoms. As used herein, C₁₋₄ alkyl refers to an alkyl selected from the group consisting of methyl, ethyl, n-propyl, iso-propyl, n-butyl, iso-butyl, sec-butyl and tert-butyl.

“Excipients” are pharmaceutically inactive substances that serve as the vehicle or medium for a drug or other active substances. In the pharmaceutical industry it is a catch-all term which includes various sub-groups comprising diluents or fillers, binders or adhesives, disintegrants, lubricants, glidants, flavors, colors, coating and sweeteners. Such components will generally be present in admixture within the formulation. The skilled person would be aware that some excipients may perform multiple functions in a formulation. For example, croscarmellose sodium when added to a formulation can act as a sweetening agent and/or a diluent. In another example, microcrystalline cellulose can act as a diluent and/or a disintegrant. Talc has been used as an anticaking agent, glidant, diluent and/or lubricant.

“Diluents” are inert substances which are able to act as fillers in the formulation. Adding a diluent to a formulation acts to make up the volume of the formulation. Due to this increase in volume, the formulation may accordingly be easier to handle.

“Binders” act to hold or draw together the different components of the formulation. In this sense, binders provide cohesive strength to the formulation. Binders can be added in a dry or wet form.

“Lubricants” are used to reduce the friction between a die wall and the formulation, preventing adhesion of the formulation to dies or punches. For example, if the formulation is to be used to form a tablet, the lubricant reduces the friction between the die wall and the formed tablet. Accordingly, the lubricant helps in allowing the tablet to be more easily ejected from the die cavity. Lubricants can be soluble or insoluble in the formulation.

“Glidants” help in the flow properties of the formulation. This is desirable as it reduces wastage and improves control as the formulation is transferred from a hopper to a die cavity, for example. Glidants acts by minimizing the friction between particles within the formulation.

“Disintegrants” are substances included in formulations to promote moisture penetration and dispersion of the matrix of the dosage form in dissolution fluids. For example, in an oral formulation, such as a tablet formulation or hard shell capsule formulation, solid dosage form should ideally disperse into the primary particles from which it was prepared.

“Anticaking agents” are also known as anti-agglomeration agents. They are used to prevent lump formation in granulation blend or in API. Agglomeration may be an issue with respect to flow, particle size and in general processability. In the presence of small amount of moisture, the API gets dissolved and the dissolved API acts as a binder and forms lumps in API itself or within the blend. The anticaking agents because of their high surface area cover the API particles and prevent caking. Additionally, the anticaking agent should not react chemically with the API or other excipients.

In a first aspect, the present invention provides a pharmaceutical oral composition. When administered orally, the pharmaceutical composition will usually be formulated into unit dosage forms such as tablets, caplets, cachets, powder, granules, beads, chewable lozenges, capsules, liquids, aqueous suspensions or solutions, or similar dosage forms, using conventional equipment and techniques known in the art. For example, the pharmaceutical oral composition may be in a liquid form or a solid form. In some embodiments, the pharmaceutical composition is in the form of a tablet. The tablet can be of any suitable size or suitable shape. In another embodiment, the pharmaceutical composition is in the form of a liquid. In another embodiment, the pharmaceutical composition is in the form of a powder. In another embodiment, the pharmaceutical composition is in the form of a capsule. In another embodiment, the pharmaceutical composition is in the form of a gel.

In some embodiments, the pharmaceutical composition comprises a compound of Formula (I) or a pharmaceutically acceptable salt, solvate or prodrug thereof:

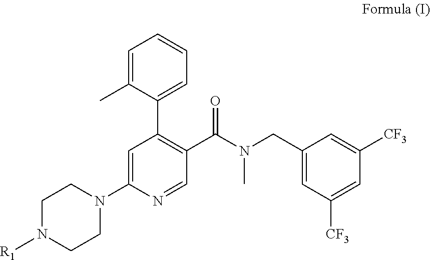

wherein R₁ is H or C₁₋₄ alkyl.

In an embodiment, R₁ is H, methyl, ethyl, n-propyl, iso-propyl, n-butyl, sec-butyl, iso-butyl or tert-butyl. In another embodiment, R₁ is H, methyl, ethyl, n-propyl or iso-propyl. In another embodiment, R₁ is H. In another embodiment, R₁ is methyl. In another embodiment, R₁ is ethyl. In another embodiment, R₁ is n-propyl. In another embodiment, R₁ is iso-propyl. In another embodiment, R₁ is n-butyl. In another embodiment, R₁ is sec-butyl. In another embodiment, R₁ is iso-butyl. In another embodiment, R₁ is tert-butyl.

Accordingly, in some embodiments, the pharmaceutical composition comprises a compound of Formula (I) or a pharmaceutically acceptable salt, solvate or prodrug thereof selected from the following:

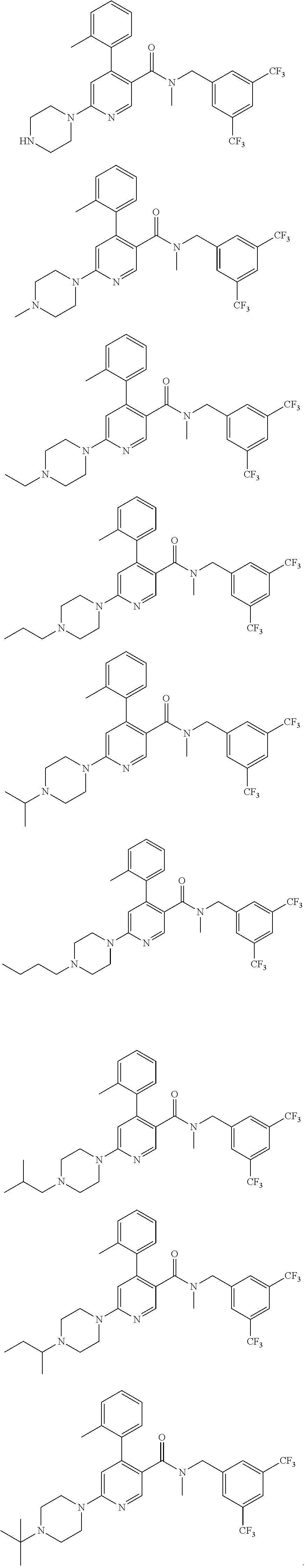

In particular, in some embodiments, the pharmaceutical composition comprises a compound of Formula (I) or a pharmaceutically acceptable salt, solvate or prodrug thereof which is:

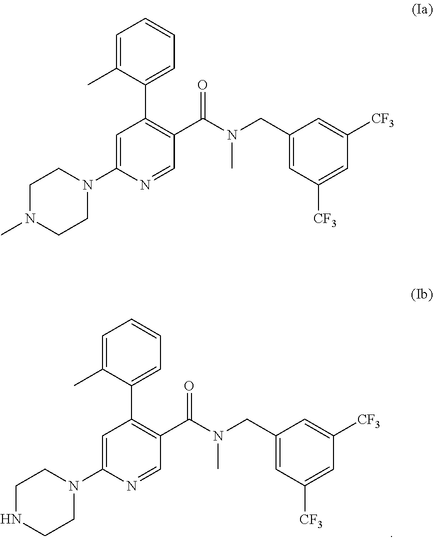 [SMILES: Cc1ccccc1-c1cc(N2CCN(C)CC2)ncc1C(=O)N(C)Cc1cc(C(F)(F)F)cc(C(F)(F)F)c1 ; Cc1ccccc1-c1cc(N2CCNCC2)ncc1C(=O)N(C)Cc1cc(C(F)(F)F)cc(C(F)(F)F)c1]

In an embodiment, compound of Formula (I) or a pharmaceutically acceptable salt, solvate or prodrug thereof is provided as a salt. In another embodiment, compound of Formula (I) or a pharmaceutically acceptable salt, solvate or prodrug thereof is a HCl salt. In another embodiment, compound of Formula (I) or a pharmaceutically acceptable salt, solvate or prodrug thereof is a 2HCl salt. Accordingly, in some embodiments, the pharmaceutical composition comprises a compound of Formula (I) or a pharmaceutically acceptable salt, solvate or prodrug thereof selected from the following:

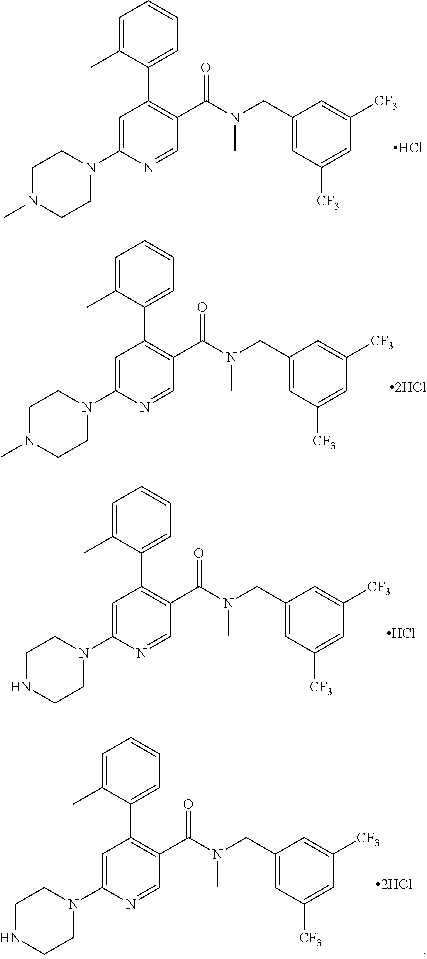

In relation to the present invention, when formulating the pharmaceutical composition, the inventors have found that a problem is the agglomeration of the API when mixed with excipients. This resulted in an undesirable appearance of the formulation, such as mottling as observed on the surface of a tablet. Another problem is that when formulating a tablet, the formulation was found to stick to the punch during compression. Further, the tablets were found to lack uniformity in the content of the API throughout batches and low recovery was also achieved. In this regard, while the inventors had experimented with multiple ways to solve the above mentioned problems. For example, various combinations and ratios of excipients were tested, but none provided a significant improvement of these above mentioned issues. Unexpectedly, the inventors have found that micronisation of the API can help to significantly alleviate these issues.

Accordingly, it is believed that the particle size of the API has a fundamental effect on both the uniformity of a formulation blend and the dissolution rate and therefore aids in the selection of suitable excipients for the final tablet formulation. As mentioned above, it was found that it was difficult to screen prior to blending for making the formulation. It was further observed that there were various coloured particles (pale yellow, orange and brown) retained on the sieve screen. Further, when the tablets were compressed, significant mottling was observed which was undesirable. It has been found that it is advantageous to have a D(0.5) particle size of the API of less than about 70 μm. In particular, when the D(0.5) particle size of the API is less than about 60 μm, the hardness of the tablet, the weight and content uniformity, and the dissolution profile are consistently maintained from batches to batches.

The skilled person would understand that a D(0.5) particle size distribution (or alternatively D(50)) refers to a particle size distribution at which 50% of the particles in the cumulative distribution intercept at the stated value. Accordingly, D(0.5) is the particle sizes cumulative distribution that 50% of a sample's particle diameter is smaller than the stated value and 50% of a sample's particle diameter is larger than the stated value. For example, if D(0.5)=5.8 μm, then 50% of the particles in the sample are larger than 5.8 μm, and 50% smaller than 5.8 μm. D(0.5) is also known as the median diameter or the medium value of the particle size distribution.

Accordingly, in some embodiments, the compound of Formula (I) or the pharmaceutically acceptable salt, solvate or prodrug thereof within the pharmaceutical composition has a D(0.5) particle size distribution of less than about 70 μm. In another embodiment, the D(0.5) is less than about 60 μm. In another embodiment, the D(0.5) is less than about 50 μm. In another embodiment, the D(0.5) is less than about 40 μm. In another embodiment, the D(0.5) is less than about 30 μm. In another embodiment, the D(0.5) is less than about 20 μm. In another embodiment, the D(0.5) is less than about 10 μm. In another embodiment, the D(0.5) is less than about 5 μm. In another embodiment, the D(0.5) is of about 5 μm to about 70 μm. In another embodiment, the D(0.5) is of about 5 μm to about 60 μm. In another embodiment, the D(0.5) is of about 10 μm to about 60 μm. In another embodiment, the D(0.5) is of about 20 μm to about 60 μm. In another embodiment, the D(0.5) is of about 30 μm to about 60 μm. In another embodiment, the D(0.5) is of about 40 μm to about 60 μm. In another embodiment, the D(0.5) is of about 5 μm to about 50 μm. In another embodiment, the D(0.5) is of about 10 μm to about 50 μm. In another embodiment, the D(0.5) is of about 20 μm to about 50 μm. In another embodiment, the D(0.5) is of about 30 μm to about 50 μm. In another embodiment, the D(0.5) is of about 40 μm to about 50 μm.

Tablets will typically include one or more excipients. Excipients should be compatible with the other ingredients of the formulation and physiologically innocuous to the recipient thereof. Examples of suitable excipients are well known to the person skilled in the art of tablet formulation and may be found e.g. in Handbook of Pharmaceutical Excipients (eds. Rowe, Sheskey & Quinn), 6th edition 2009.

In an embodiment, the pharmaceutical composition comprises at least one diluent. The diluent may be selected from the group consisting of lactose, sorbitol, dibasic calcium phosphate dihydrate, calcium sulphate dihydrate, calcium carbonate, croscarmellose sodium, calcium phosphate, calcium hydrogen phosphate dihydrate, crospovidone, ferric oxide, magnesium carbonate, magnesium oxide, sucrose, or sodium chloride. In another embodiment, the at least one diluent may be selected from the group consisting of lactose, sorbitol, croscarmellose sodium, crospovidone, ferric oxide, magnesium carbonate, magnesium oxide, or sucrose. In another embodiment, the at least one diluent may be selected from the group consisting of lactose, sorbitol, or sucrose. In another embodiment, the at least one diluent is lactose. Lactose can be used in anhydrous or hydrated form (e.g. monohydrate), and is typically prepared by spray drying, fluid bed granulation, or roller drying. In another embodiment, the at least one diluent is sorbitol. In another embodiment, the at least one diluent is sucrose.

In an embodiment, the at least one diluent is present in the composition in an amount from about 10% to about 90% wt/wt based on the total weight of the composition. In another embodiment, the at least one diluent is present in the composition in an amount from about 20% to about 80% wt/wt based on the total weight of the composition. In another embodiment, the at least one diluent is present in the composition in an amount from about 30% to about 70% wt/wt based on the total weight of the composition. In another embodiment, the at least one diluent is present in the composition in an amount from about 35% to about 70% wt/wt based on the total weight of the composition. In another embodiment, the at least one diluent is present in the composition in an amount from about 40% to about 70% wt/wt based on the total weight of the composition. In another embodiment, the at least one diluent is present in the composition in an amount from about 45% to about 70% wt/wt based on the total weight of the composition. In another embodiment, the at least one diluent is present in the composition in an amount from about 50% to about 70% wt/wt based on the total weight of the composition.

In an embodiment, the pharmaceutical composition comprises at least one lubricant. The lubricant may be selected from the group consisting of magnesium stearate, stearic acid, calcium stearate, paraffin, sodium lauryl sulphate, sodium benzoate, castor oil hydrogenated, glyceryl monostearate, glyceryl behenate, sodium stearyl fumarate, mineral oil, polaxamer, PEG 400, PEG 600, or PEG 8000. In another embodiment, the at least one lubricant may be selected from the group consisting of magnesium stearate, stearic acid, calcium stearate, sodium lauryl sulphate, sodium benzoate, glyceryl monostearate, sodium stearyl fumarate, polaxamer, PEG 400, PEG 600, or PEG 8000. In another embodiment, the at least one lubricant may be selected from the group consisting of magnesium stearate, stearic acid, calcium stearate, PEG 400, PEG 600, or PEG 8000. In another embodiment, the lubricant is magnesium stearate. In another embodiment, the lubricant is stearic acid. In another embodiment, the lubricant is calcium stearate. In another embodiment, the lubricant is PEG 400. In another embodiment, the lubricant is PEG 600. In another embodiment, the lubricant is PEG 8000.

In an embodiment, the at least one lubricant is present in the composition in an amount from about 0.01% to about 4% wt/wt based on the total weight of the composition. In another embodiment, the at least one lubricant is present in the composition in an amount from about 0.05% to about 3.5% wt/wt based on the total weight of the composition. In another embodiment, the at least one lubricant is present in the composition in an amount from about 0.1% to about 3% wt/wt based on the total weight of the composition. In another embodiment, the at least one lubricant is present in the composition in an amount from about 0.1% to about 2.5% wt/wt based on the total weight of the composition. In another embodiment, the at least one lubricant is present in the composition in an amount from about 0.1% to about 2% wt/wt based on the total weight of the composition. In another embodiment, the at least one lubricant is present in the composition in an amount from about 0.3% to about 2% wt/wt based on the total weight of the composition. In another embodiment, the at least one lubricant is present in the composition in an amount from about 0.5% to about 2% wt/wt based on the total weight of the composition. In another embodiment, the at least one lubricant is present in the composition in an amount from about 0.7% to about 2% wt/wt based on the total weight of the composition. In another embodiment, the at least one lubricant is present in the composition in an amount from about 1% to about 2% wt/wt based on the total weight of the composition.

In an embodiment, the pharmaceutical composition comprises at least one disintegrant. The disintegrant may be selected from the group consisting of microcrystalline cellulose, alginic acid, citric acid, croscamellose sodium, carboxy methyl cellulose calcium, cysteine HCl, methyl cellulose, polyoxy stearate, sodium starch glycolate, sodium alginate, or carboxy methyl cellulose sodium. In another embodiment, the at least one disintegrant may be selected from the group consisting of microcrystalline cellulose, alginic acid, citric acid, croscamellose sodium, carboxy methyl cellulose calcium, cysteine HCl, methyl cellulose, polyoxy stearate, sodium starch glycolate, or carboxy methyl cellulose sodium. In another embodiment, the at least one disintegrant may be selected from the group consisting of microcrystalline cellulose, sodium starch glycolate, carboxy methyl cellulose calcium, methyl cellulose, or carboxy methyl cellulose sodium. In another embodiment, the at least one disintegrant may be selected from the group consisting of microcrystalline cellulose, carboxy methyl cellulose calcium, methyl cellulose, or carboxy methyl cellulose sodium. In another embodiment, the disintegrant is microcrystalline cellulose. In another embodiment, the disintegrant is sodium starch glycolate. In another embodiment, the disintegrant is carboxy methyl cellulose calcium. In another embodiment, the disintegrant is methyl cellulose. In another embodiment, the disintegrant is carboxy methyl cellulose sodium.

In an embodiment, the at least one disintegrant is present in the composition in an amount from about 10% to about 40% wt/wt based on the total weight of the composition. In another embodiment, the at least one disintegrant is present in the composition in an amount from about 15% to about 35% wt/wt based on the total weight of the composition. In another embodiment, the at least one disintegrant is present in the composition in an amount from about 18% to about 33% wt/wt based on the total weight of the composition. In another embodiment, the at least one disintegrant is present in the composition in an amount from about 20% to about 30% wt/wt based on the total weight of the composition. In another embodiment, the at least one disintegrant is present in the composition in an amount from about 22% to about 28% wt/wt based on the total weight of the composition.

In an embodiment, when the disintegrant is microcrystalline cellulose, the disintegrant is present in the composition in an amount from about 10% to about 40% wt/wt based on the total weight of the composition. In another embodiment, the disintegrant is present in the composition in an amount from about 15% to about 35% wt/wt based on the total weight of the composition. In another embodiment, the disintegrant is present in the composition in an amount from about 18% to about 33% wt/wt based on the total weight of the composition. In another embodiment, the disintegrant is present in the composition in an amount from about 20% to about 30% wt/wt based on the total weight of the composition. In another embodiment, the disintegrant is present in the composition in an amount from about 22% to about 28% wt/wt based on the total weight of the composition.

In an embodiment, when the disintegrant is sodium starch glycolate, the disintegrant is present in the composition in an amount from about 2% to about 7% wt/wt based on the total weight of the composition. In another embodiment, the disintegrant is present in the composition in an amount from about 2% to about 6.5% wt/wt based on the total weight of the composition. In another embodiment, the disintegrant is present in the composition in an amount from about 2% to about 6% wt/wt based on the total weight of the composition. In another embodiment, the disintegrant is present in the composition in an amount from about 2.5% to about 5.5% wt/wt based on the total weight of the composition. In another embodiment, the disintegrant is present in the composition in an amount from about 3% to about 5% wt/wt based on the total weight of the composition. In another embodiment, the disintegrant is present in the composition in an amount from about 3% to about 4.5% wt/wt based on the total weight of the composition. In another embodiment, the disintegrant is present in the composition in an amount from about 3% to about 4% wt/wt based on the total weight of the composition.

In an embodiment, the pharmaceutical composition comprises at least one binder. The binder may be selected from the group consisting of starch, gelatin, glucose, polyvinyl pyrrolidone (Povidone), carboxymethyl cellulose, acacia, candelilla wax, carnuba wax, cornstarch, glyceryl behenate, hypromellose, or polyethylene oxide. In another embodiment, the at least one binder may be selected from the group consisting of starch, gelatin, glucose, polyvinyl pyrrolidone (Povidone), acacia, candelilla wax, carnuba wax, cornstarch, glyceryl behenate, or hypromellose. In another embodiment, the at least one binder may be selected from the group consisting of starch, gelatin, glucose, acacia, candelilla wax, carnuba wax, or cornstarch. In another embodiment, the binder is starch. In another embodiment, the binder is gelatin. In another embodiment, the binder is glucose. In another embodiment, the binder is acacia. In another embodiment, the binder is candelilla wax. In another embodiment, the binder is carnuba wax. In another embodiment, the binder is cornstarch.

In an embodiment, the at least one binder is present in the composition in an amount from about 2% to about 20% wt/wt based on the total weight of the composition. In another embodiment, the at least one binder is present in the composition in an amount from about 3% to about 19% wt/wt based on the total weight of the composition. In another embodiment, the at least one binder is present in the composition in an amount from about 4% to about 18% wt/wt based on the total weight of the composition. In another embodiment, the at least one binder is present in the composition in an amount from about 5% to about 17% wt/wt based on the total weight of the composition. In another embodiment, the at least one binder is present in the composition in an amount from about 5% to about 16% wt/wt based on the total weight of the composition. In another embodiment, the at least one binder is present in the composition in an amount from about 5% to about 15% wt/wt based on the total weight of the composition. In another embodiment, the at least one binder is present in the composition in an amount from about 5% to about 14% wt/wt based on the total weight of the composition. In another embodiment, the at least one binder is present in the composition in an amount from about 5% to about 13% wt/wt based on the total weight of the composition. In another embodiment, the at least one binder is present in the composition in an amount from about 5% to about 12% wt/wt based on the total weight of the composition. In another embodiment, the at least one binder is present in the composition in an amount from about 5% to about 11% wt/wt based on the total weight of the composition. In another embodiment, the at least one binder is present in the composition in an amount from about 5% to about 10% wt/wt based on the total weight of the composition.

As mentioned above, the issues of mottling, sticking to the punch and consistency of the tablet batches can be alleviated by the micronisation of the API. It was found that a combination of micronisation of the API and use of an anticaking agent can further alleviate these issues. In particular, a combination of micronisation of the API and use of specific anticaking agent can produce a tablet formulation with no mottling, does not stick to the punch and is consistent from batches to batches.

Accordingly, in an embodiment, the at least one anti-caking agent may be selected from the group consisting of fumed silica, silicon dioxide, or talc. In another embodiment, the anti-caking agent is fumed silica. In another embodiment, the anti-caking agent is silicon dioxide. In another embodiment, the anti-caking agent is talc.

In an embodiment, the at least one anti-caking agent is present in the composition in an amount from about 0.05% to about 4% wt/wt based on the total weight of the composition. In another embodiment, the at least one anti-caking agent is present in the composition in an amount from about 0.1% to about 3.5% wt/wt based on the total weight of the composition. In another embodiment, the at least one anti-caking agent is present in the composition in an amount from about 0.15% to about 3% wt/wt based on the total weight of the composition. In another embodiment, the at least one anti-caking agent is present in the composition in an amount from about 0.2% to about 2.5% wt/wt based on the total weight of the composition. In another embodiment, the at least one anti-caking agent is present in the composition in an amount from about 0.2% to about 2% wt/wt based on the total weight of the composition. In another embodiment, the at least one anti-caking agent is present in the composition in an amount from about 0.25% to about 2% wt/wt based on the total weight of the composition. In another embodiment, the at least one anti-caking agent is present in the composition in an amount from about 0.3% to about 2% wt/wt based on the total weight of the composition. In another embodiment, the at least one anti-caking agent is present in the composition in an amount from about 0.35% to about 2% wt/wt based on the total weight of the composition. In another embodiment, the at least one anti-caking agent is present in the composition in an amount from about 0.4% to about 2% wt/wt based on the total weight of the composition. In another embodiment, the at least one anti-caking agent is present in the composition in an amount from about 0.45% to about 2% wt/wt based on the total weight of the composition. In another embodiment, the at least one anti-caking agent is present in the composition in an amount from about 0.5% to about 2% wt/wt based on the total weight of the composition.

Tablets provided herein may be uncoated or coated (in which case they include a coating). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water soluble material included in the film coating of the present invention may include a single polymer material, it may also be formed using a mixture of more than one polymer. The coating may be white or coloured e.g. gray. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional colouring such as iron oxide or indigo carmine or iron oxide yellow or FD&C yellow #6). The amount of coating will generally be between about 2-4% of the core's weight, and in certain specific embodiments, about 3%. Unless specifically stated otherwise, where the dosage form is coated, it is to be understood that a reference to % weight of the tablet means that of the total tablet, i.e. including the coating.

Accordingly, the pharmaceutical composition may further comprise at least one coating selected from the group consisting of hydroxypropyl cellulose, hydroxypropyl methylcellulose, hydroxypropyl methyl cellulose phthalate, methyl cellulose, methacrylic acid copolymer, erthrosine sodium, or sodium propionate. In another embodiment, the at least one coating may be selected from hydroxypropyl cellulose, hydroxypropyl methylcellulose, hydroxypropyl methyl cellulose phthalate, methyl cellulose, or methacrylic acid copolymer. In another embodiment, the coating is hydroxypropyl cellulose. In another embodiment, the coating is hydroxypropyl methylcellulose. In another embodiment, the coating is hydroxypropyl methyl cellulose phthalate. In another embodiment, the coating is methyl cellulose. In another embodiment, the coating is methacrylic acid copolymer.

Suitable pharmaceutical compositions contain, e.g., from about 0.1% to about 99.9%, of compound of Formula (I) or the pharmaceutically acceptable salt, solvate or prodrug thereof (active ingredient). In an embodiment, the active ingredient may be present in the composition in an amount from about 0.1% to about 90% wt/wt based on the total weight of the composition. In another embodiment, the active ingredient may be present in the composition in an amount from about 0.1% to about 80% wt/wt based on the total weight of the composition. In another embodiment, the active ingredient may be present in the composition in an amount from about 0.1% to about 70% wt/wt based on the total weight of the composition. In another embodiment, the active ingredient may be present in the composition in an amount from about 0.1% to about 60% wt/wt based on the total weight of the composition. In another embodiment, the active ingredient may be present in the composition in an amount from about 0.1% to about 50% wt/wt based on the total weight of the composition. In another embodiment, the active ingredient may be present in the composition in an amount from about 1% to about 80% wt/wt based on the total weight of the composition. In another embodiment, the active ingredient may be present in the composition in an amount from about 5% to about 80% wt/wt based on the total weight of the composition. In another embodiment, the active ingredient may be present in the composition in an amount from about 10% to about 80% wt/wt based on the total weight of the composition. In another embodiment, the active ingredient may be present in the composition in an amount from about 15% to about 80% wt/wt based on the total weight of the composition. In another embodiment, the active ingredient may be present in the composition in an amount from about 20% to about 80% wt/wt based on the total weight of the composition.

In some embodiments, the pharmaceutical composition in the form of a tablet comprises:

(i) compound of Formula (I) or or a pharmaceutically acceptable salt, solvate or prodrug thereof as described herein;

(ii) lactose;

(iii) magnesium stearate;

(iv) microcrystalline cellulose;

(v) starch; and

(vi) sodium starch glycolate.

In other embodiments, the pharmaceutical composition in the form of a tablet comprises:

(i) a compound of Formula (I) or a pharmaceutically acceptable salt, solvate or prodrug thereof as described herein; wherein the compound of Formula (I) or the pharmaceutically acceptable salt, solvate or prodrug thereof within the composition has a D(0.5) particle size distribution of less than 60 μm;

(ii) lactose;

(iii) magnesium stearate;

(iv) microcrystalline cellulose;

(v) starch; and

(vi) sodium starch glycolate.

In other embodiments, the pharmaceutical composition in the form of a tablet comprises:

(i) compound of Formula (I) or or a pharmaceutically acceptable salt, solvate or prodrug thereof as described herein; wherein the compound of Formula (I) or the pharmaceutically acceptable salt, solvate or prodrug thereof within the composition has a D(0.5) particle size distribution of less than 60 μm;

(ii) lactose, wherein lactose is present in the composition in an amount from about 35% to about 70% wt/wt based on the total weight of the composition;

(iii) magnesium stearate; wherein magnesium stearate is present in the composition in an amount from about 0.1% to about 2% wt/wt based on the total weight of the composition;

(iv) microcrystalline cellulose, wherein microcrystalline cellulose is present in the composition in an amount from about 20% to about 30% wt/wt based on the total weight of the composition;

(v) starch, wherein starch is present in the composition in an amount from about 5% to about 15% wt/wt based on the total weight of the composition; and

(vi) sodium starch glycolate, wherein sodium starch glycolate is present in the composition in an amount from about 2% to about 7% wt/wt based on the total weight of the composition.

In some embodiments, the pharmaceutical composition comprises:

(i) compound of Formula (I) or or a pharmaceutically acceptable salt, solvate or prodrug thereof as described herein;

(ii) lactose;

(iii) magnesium stearate;

(iv) microcrystalline cellulose;

(v) starch; and

(vi) fumed silica.

In some embodiments, the pharmaceutical composition in the form of a tablet comprises:

(i) a compound of Formula (I) or a pharmaceutically acceptable salt, solvate or prodrug thereof as described herein, wherein the compound of Formula (I) or the pharmaceutically acceptable salt, solvate or prodrug thereof within the composition has a D(0.5) particle size distribution of less than 60 μm;

(ii) lactose;

(iii) magnesium stearate;

(iv) microcrystalline cellulose;

(v) starch; and

(vi) fumed silica.

In other embodiments, the pharmaceutical composition in the form of a tablet comprises:

(i) a compound of Formula (I) or a pharmaceutically acceptable salt, solvate or prodrug thereof as described herein; wherein the compound of Formula (I) or the pharmaceutically acceptable salt, solvate or prodrug thereof within the composition has a D(0.5) particle size distribution of less than 60 μm;

(ii) lactose, wherein lactose is present in the composition in an amount from about 35% to about 70% wt/wt based on the total weight of the composition;

(iii) magnesium stearate, wherein magnesium stearate is present in the composition in an amount from about 0.1% to about 2% wt/wt based on the total weight of the composition;

(iv) microcrystalline cellulose, wherein microcrystalline cellulose is present in the composition in an amount from about 20% to about 30% wt/wt based on the total weight of the composition;

(v) starch, wherein starch is present in the composition in an amount from about 5% to about 15% wt/wt based on the total weight of the composition; and

(vi) fumed silica, wherein fumed silica is present in the composition in an amount from about 0.2% to about 2% wt/wt based on the total weight of the composition.

Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid.

Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of a compound with the appropriate inorganic or organic acid via any of a number of known methods. Alternatively, alkali and alkaline earth metal salts can be prepared by reacting a compound with the appropriate base via a variety of known methods. The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

It should be understood that in addition to the ingredients particularly mentioned above, the compositions of this invention may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include aspartame or saccharine. Suitable flavouring agents include peppermint oil, oil of wintergreen, cherry, orange or raspberry flavouring. Suitable preservatives include sodium benzoate, vitamin E, alpha-tocopherol, ascorbic acid, methyl paraben, propyl paraben or sodium bisulphite. Suitable time delay agents include glyceryl monostearate or glyceryl distearate.

The composition may contain any other suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

For example, the pharmaceutical composition may further comprise a preservative, a buffer, stabiliser and/or a viscosity enhancing agent. Examples of suitable preservatives are benzoic acid esters of para-hydroxybenzoic acid, propylene glycol, phenols, phenylethyl alchohol or benzyl alcohol. Examples of suitable buffers are sodium phosphate salts, citric acid, tartaric acid and the like. Examples of suitable stabilisers are, antioxidants such as alpha-tocopherol acetate, alpha-thioglycerin, sodium metabisulphite, ascorbic acid, acetylcysteine, 8-hydroxyquinoline, chelating agents such as disodium edetate. Examples of suitable viscosity enhancing agents, suspending or dispersing agents are polyvinyl alcohol, carbomer, polyoxypropylene glycols, sorbitan monooleate, sorbitan sesquioleate, polyoxyethylene hydrogenated castor oil 60.

For example, the pharmaceutical composition may further comprise a pH controller and/or an isotonic agent. Examples of suitable pH controllers include hydrochloric acid, sodium hydroxide and the like. Examples of suitable isotonic agents are glucose, D-sorbitol or D-mannitol, sodium chloride.

The carrier must be pharmaceutically “acceptable” in the sense of being compatible with the other ingredients of the composition and not injurious to the subject. Compositions include those suitable for oral, rectal, nasal, topical (including buccal and sublingual), vaginal or parental (including subcutaneous, intramuscular, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

In some embodiments, it was observed that the pharmaceutical composition was sticking to the punch tablet press during compression into tablet. Advantageously, it was observed that by varying the nominal weight of the tablet, the sticking effect may be further minimised and/or eliminated.

Accordingly, in an embodiment, the pharmaceutical composition, in the form of a tablet, has a weight of about 50 mg to about 500 mg. In another embodiment, the tablet is of about 50 mg to about 450 mg. In another embodiment, the tablet is of about 50 mg to about 400 mg. In another embodiment, the tablet is of about 50 mg to about 350 mg. In another embodiment, the tablet is of about 50 mg to about 300 mg. In another embodiment, the tablet is of about 100 mg to about 300 mg. In another embodiment, the tablet is of about 50 mg. In another embodiment, the tablet is of about 75 mg. In another embodiment, the tablet is of about 100 mg. In another embodiment, the tablet is of about 150 mg. In another embodiment, the tablet is of about 200 mg. In another embodiment, the tablet is of about 250 mg. In another embodiment, the tablet is of about 300 mg. In another embodiment, the tablet is of about 350 mg. In another embodiment, the tablet is of about 400 mg. In another embodiment, the tablet is of about 450 mg. In another embodiment, the tablet is of about 500 mg.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum;

pastilles comprising the active ingredient in an inert basis such as gelatine and glycerin, or sucrose and acacia gum; and mouthwashes comprising the active ingredient in a suitable liquid carrier.

Preferred unit dosage compositions are those containing a daily dose or unit, daily sub-dose, as herein above described, or an appropriate fraction thereof, of the active ingredient.

In an embodiment, the pharmaceutical composition in the form of a tablet is an immediate release pharmaceutical composition. In this regard, the pharmaceutical composition is formulated to release the API immediately after oral administration. Immediate-release products generally result in relatively rapid drug absorption and onset of accompanying pharmacodynamic effects. It is advantageous for the present pharmaceutical composition to provide rapid relief of elevated ICP to a subject in need thereof as the condition of the subject may deteriorate the longer he/she is in this unusual state. Elevated ICP is very likely to cause severe harm and is usually fatal if prolonged. For example, elevated ICP may crush brain tissue, shift brain structures, contribute to hydrocephalus, cause brain herniation, and restrict blood supply to the brain. Accordingly, the present composition may provide relief from condition and/or symptom resulting from elevated ICP such as, but not limited to, headache, vomiting without nausea, ocular palsies, altered level of consciousness, increased blood pressure, back pain, double vision, papilledema or further injury to the brain or spinal cord, or a combination thereof.

In an embodiment, compound of Formula (I) is released immediately after oral administration. In another embodiment, compound of Formula (I) is released 1 min after oral administration. In another embodiment, compound of Formula (I) is released 5 min after oral administration. In another embodiment, compound of Formula (I) is released 10 min after oral administration. In another embodiment, compound of Formula (I) is released 15 min after oral administration. In another embodiment, compound of Formula (I) is released 20 min after oral administration. In another embodiment, compound of Formula (I) is released 25 min after oral administration. In another embodiment, compound of Formula (I) is released 30 min after oral administration. In another embodiment, compound of Formula (I) is released 40 min after oral administration. In another embodiment, compound of Formula (I) is released 50 min after oral administration. In another embodiment, compound of Formula (I) is released 60 min after oral administration. In another embodiment, compound of Formula (I) is released 90 min after oral administration.

In a second aspect, the present invention provides a method for treating elevated intracranial pressure in a subject in need thereof, the method comprising administering to the subject a pharmaceutical composition in the form of a tablet comprising:
(i) a compound of Formula (I) or a pharmaceutically acceptable salt, solvate or prodrug thereof:

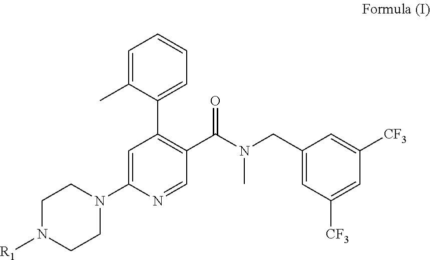

wherein R₁ is H or C₁₋₄ alkyl; and
wherein the compound of Formula (I) or the pharmaceutically acceptable salt, solvate or prodrug thereof within the composition has a D(0.5) particle size distribution of less than about 60 μm;
(ii) at least one diluent selected from the group consisting of lactose, sorbitol, dibasic calcium phosphate dihydrate, calcium sulphate dihydrate, calcium carbonate, croscarmellose sodium, calcium phosphate, calcium hydrogen phosphate dihydrate, crospovidone, ferric oxide, magnesium carbonate, magnesium oxide, sucrose, or sodium chloride, wherein the at least one diluent is present in the composition in an amount from about 35% to about 70% wt/wt based on the total weight of the composition;
(iii) at least one lubricant selected from the group consisting of magnesium stearate, stearic acid, calcium stearate, paraffin, sodium lauryl sulphate, sodium benzoate, castor oil hydrogenated, glyceryl monostearate, glyceryl behenate, sodium stearyl fumarate, mineral oil, polaxamer, PEG 400, PEG 600, or PEG 8000, wherein the at least one lubricant is present in the composition in an amount from about 0.1% to about 2% wt/wt based on the total weight of the composition;
(iv) at least one disintegrant selected from the group consisting of microcrystalline cellulose, alginic acid, citric acid, croscamellose sodium, carboxy methyl cellulose calcium, cysteine HCl, methyl cellulose, polyoxy stearate, sodium starch glycolate, sodium alginate, or carboxy methyl cellulose sodium, wherein the at least one disintegrant is present in the composition in an amount from about 20% to about 30% wt/wt based on the total weight of the composition;
(v) at least one binder selected from the group consisting of starch, gelatin, glucose, polyvinyl pyrrolidone (Povidone), carboxymethylcellulose, acacia, candelilla wax, carnuba wax, cornstarch, glyceryl behenate, hypromellose, or polyethylene oxide, wherein the at least one binder is present in the composition in an amount from about 5% to about 15% wt/wt based on the total weight of the composition; and
(vi) at least one anti-caking agent selected from the group consisting of fumed silica, silicon dioxide, or talc, wherein the at least one anti-caking agent is present in the composition in an amount from about 0.2% to about 2% wt/wt based on the total weight of the composition.

In a third aspect, the present invention provides a pharmaceutical composition in the form of a tablet for use in the treatment of elevated intracranial pressure in a subject in need thereof, the pharmaceutical composition is as described herein.

In one embodiment, the dosage of the pharmaceutical composition administered to a subject in the various embodiments of the present invention is such that compound of Formula (I) is administered in the range from 0.1 mg/kg to 100 mg/kg. In one embodiment, the dosage of the pharmaceutical composition administered to a subject in the various embodiments of the present invention is such that compound of Formula (I) is administered in the range from 0.1 mg/kg to 100 mg/kg. For instance, the dosage amount may be 0.1 mg/kg, 0.2 mg/kg, 0.5 mg/kg, 1.0 mg/kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10.0 mg/kg, 11.0 mg/kg, 12.0 mg/kg, 13.0 mg/kg, 14.0 mg/kg, 15.0 mg/kg, 16.0 10 mg/kg, 17.0 mg/kg, 18.0 mg/kg, 19.0 mg/kg, 20.0 mg/kg, 21.0 mg/kg, 22.0 mg/kg, 23.0 mg/kg, 24.0 mg/kg, 25.0 mg/kg, 26.0 mg/kg, 27.0 mg/kg, 28.0 mg/kg, 29.0 mg/kg, 30.0 mg/kg, 31.0 mg/kg, 32.0 mg/kg, 33.0 mg/kg, 34.0 mg/kg, 35.0 mg/kg, 36.0 mg/kg, 37.0 mg/kg, 38.0 mg/kg, 39.0 mg/kg, 40.0 mg/kg, 41.0 mg/kg, 42.0 mg/kg, 43.0 mg/kg, 44.0 mg/kg, 45.0 mg/kg, 46.0 mg/kg, 47.0 mg/kg, 48.0 mg/kg, 49.0 mg/kg, 50.0 mg/kg, 51.0 mg/kg, 52.0 mg/kg, 53.0 mg/kg, 54.0 mg/kg, 55.0 mg/kg, 56.0 mg/kg, 57.0 mg/kg, 58.0 mg/kg, 59.0 mg/kg, 60.0 mg/kg, 61.0 mg/kg, 62.0 mg/kg, 63.0 mg/kg, 64.0 mg/kg, 65.0 mg/kg, 66.0 mg/kg, 67.0 mg/kg, 68.0 mg/kg, 69.0 mg/kg, 70.0 mg/kg, 71.0 mg/kg, 72.0 mg/kg, 73.0 mg/kg, 74.0 mg/kg, 75.0 mg/kg, 76.0 mg/kg, 77.0 mg/kg, 78.0 mg/kg, 79.0 mg/kg, 80.0 mg/kg, 81.0 mg/kg, 82.0 mg/kg, 83.0 mg/kg, 84.0 mg/kg, 85.0 mg/kg, 86.0 mg/kg, 87.0 mg/kg, 88.0 mg/kg, 89.0 mg/kg, 90.0 mg/kg, 91.0 mg/kg, 92.0 mg/kg, 93.0 mg/kg, 94.0 mg/kg, 95.0 mg/kg, 96.0 mg/kg, 97.0 mg/kg, 98.0 mg/kg, or 99.0 mg/kg.

In an embodiment the pharmaceutical composition shall be administered as a treatment for injury associated with concussion post the injury event.

In an embodiment, the method for treating elevated intracranial pressure is a method for treating traumatic brain injury.

In another embodiment, the method for treating elevated intracranial pressure is a method for treating stroke.

In an embodiment the effective amount is an amount which is able to maintain the blood concentration of the compound of formula (I), or a pharmaceutically acceptable salt, solvate, or prodrug thereof, in the therapeutic range for at least 3 days, for instance at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days.

In an embodiment the effective amount is administered as a single or multiple dose. In an embodiment the effective amount is administered as a single or multiple oral dose.

The terms “treat,” “treatment,” and “treating” refer to one or more of the following:

(a) relieving or alleviating at least one symptom of a disorder in a subject, including for example, reducing intracranial pressure in a TBI patient or preventing PCS after a concussion;

(b) relieving or alleviating the intensity and/or duration of a manifestation of a disorder experienced by a subject including, but not limited to, those that are in response to a given stimulus (e.g., pressure, tissue injury, cold temperature, etc.); and

(c) arresting, delaying the onset (i.e., the period prior to clinical manifestation of a disorder) and/or reducing the risk of developing or worsening a disorder.

A subject or patient in whom administration of the therapeutic compound is an effective therapeutic regimen for a disease or disorder is preferably a human.

It will be appreciated that any compound that is a prodrug of a compound of formula (I) is also within the scope and spirit of the invention. The term “pro-drug” is used in its broadest sense and encompasses those derivatives that are converted in vivo to the compounds of the invention. Such derivatives would readily occur to those skilled in the art, and include, for example, phosphonic acid derivatives.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Pre-Formulation Study

Characterisation of Active Pharmaceutical Ingredient (API)

Compound (Ia) HCl (API) is an acidic compound with an approximate pH of 2.5. Compound of Formula (I), in particular compound (Ia) as shown below, is used in all examples, and in particular the 2HCl salt of compound (Ia) (Compound (Ia) 2HCl).

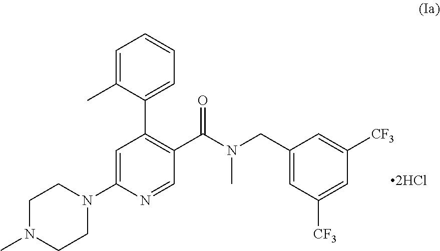 [SMILES: Cc1ccccc1-c1cc(N2CCN(C)CC2)ncc1C(=O)N(C)Cc1cc(C(F)(F)F)cc(C(F)(F)F)c1]

Particle Size

The particle size distribution of compound (Ia) 2HCl (API) was determined using a Malvern Mastersizer. The results obtained are summarised in Table 1. D(0.1) means that 10% of the sample is present as particles smaller than this size. D(0.5) means that 50% of the sample is present as particles smaller than this size. D(0.9) means that 90% of the sample is present as particles smaller than this size.

TABLE 1
Particle Size Distribution of Compound (Ia)
Particle Size (μm)

| D (0.1) | D (0.5) | D (0.9) |
|---|---|---|
| 8.41 | 76.13 | 404.59 |

Bulk and Tap Density

Measurement of the bulk and tap density provides information on the flow properties and compressibility of the API. The bulk density is measured as the density of the material as it is “poured” or passively filled into a measuring vessel, whereas the tap density is a limiting density attained after “tapping in” the material. Bulk and tap density and the subsequent Carr's index were determined on Compound (Ia) HCl and the results are given in Table 2. A Carr's index greater than 25 is considered to be an indication of poor flowability, and below 15, of good flowability.

TABLE 2
Bulk and Tap Density of Compound (Ia) 2HCl API

| | Bulk Density | Tap Density | Carr's Index |
|---|---|---|---|
| Sample | (g/mL) | (g/mL) | (%) |
| Compound (Ia) (HCl) | 0.63 | 0.83 | 24.29 |

Solubility and Intrinsic Dissolution Rate (IDR)

The solubility and IDR provides useful information the pre-formulation and characterization of solid dosage forms consisting of bulk drug substances and excipients and highlights potential bio absorption problems.

Excipient Compatibility

The successful formulation of a stable and effective solid dosage form depends on careful selection of excipients that are added to facilitate administration, promote consistent release, aid in the manufacture and protect it from degradation. Excipient compatibility is investigated by subjecting a series of blends containing approximately 50:50 API: excipient to elevated temperature for a nominated period and monitoring any observed degradation.

TABLE 3
Excipients

| Material | Function |
|---|---|
| Lactose DC | Filler, binder |
| Microcrystalline Cellulose | Filler, disintegrant |
| (Avicel PH-101) | |
| Glucose | Filler |
| Magnesium Stearate | Lubricant |
| Croscarmellose Sodium | Disintegrant |
| Starch | Binder |
| Colloidal Silicon Dioxide | Glidant/disintegrant |
| (Aerosil 200 Pharma) | |
| Sodium Lauryl Sulfate | Lubricant |
| Sodium Starch Glycolate | Disintegrant |
| (Explotab) | |
| Povidone BP 30 | Disintegrant, binder |
| Mannitol | Filler |
| Talc (Extra Fine USP) | Glidant, filler, lubricant |

The materials detailed in Table 3 were treated as follows:

1. 400 mg of excipient was dry blended with 400 mg Compound 1(a) 2HCl in duplicate.

2. The blend obtained was placed into a glass vial and sealed with a screw cap lid.

3. One sealed container was placed in a 25° C./60% RH incubator and the other sealed container was placed in a 40° C./75% RH incubator for 4 weeks.

After the 4 week storage period, the samples were analysed. All samples analysed for related substances were compared to the initial results (Time=0). The results are presented in Tables 4 to 6.

TABLE 4
% Assay Results for Each Excipient Combination

| | | After 4 weeks | After 4 weeks |
|---|---|---|---|
| Excipient | Initial | at 25° C./60% | at 40° C./75% |
| Description | (Area %) | RH (Area %) | RH (Area %) |
| Lactose | 98.44 | 99.15 | 99.23 |
| Microcrystalline | 98.44 | 99.19 | 99.17 |
| Cellulose | | | |
| Glucose | 98.40 | 99.19 | 99.17 |
| Magnesium Stearate | 98.84 | 99.15 | 99.11 |
| Croscarmellose Sodium | 98.53 | 99.20 | 99.11 |
| Starch | 98.51 | 99.14 | 99.10 |
| Colloidal Silicon | 98.53 | 99.15 | 99.11 |
| Dioxide | | | |
| Sodium Lauryl Sulfate | 98.56 | 99.18 | 99.10 |
| Sodium Starch | 98.57 | 99.28 | 99.10 |
| Glycolate | | | |
| Povidone | 98.59 | 98.98 | 98.90 |
| Mannitol | 98.51 | 99.13 | 99.09 |
| Talc | 98.58 | 99.18 | 99.08 |

TABLE 5
% Major Impurity Results for Excipient Combination

| | | After 4 weeks | After 4 weeks |
|---|---|---|---|
| Excipient | Initial | at 25° C./60% | at 40° C./75% |
| Description | (Area %) | RH (Area %) | RH (Area %) |
| Lactose | 0.57 | 0.56 | 0.57 |
| Microcrystalline | 0.57 | 0.56 | 0.57 |
| Cellulose | | | |
| Glucose | 0.57 | 0.55 | 0.57 |
| Magnesium Stearate | 0.56 | 0.54 | 0.56 |
| Croscarmellose Sodium | 0.55 | 0.54 | 0.57 |
| Starch | 0.57 | 0.56 | 0.57 |
| Colloidal Silicon | 0.57 | 0.55 | 0.56 |
| Dioxide | | | |
| Sodium Lauryl Sulfate | 0.56 | 0.56 | 0.57 |
| Sodium Starch | 0.55 | 0.48 | 0.56 |
| Glycolate | | | |
| Povidone | 0.57 | 0.57 | 0.58 |
| Mannitol | 0.57 | 0.56 | 0.58 |
| Talc | 0.57 | 0.55 | 0.57 |

TABLE 6
% Total Impurities Results for Excipient Combination

| | | After 4 weeks at | After 4 weeks at |
|---|---|---|---|
| Excipient | Initial | 25° C./60% | 40° C./75% |
| Description | (Area %) | RH (Area %) | RH (Area %) |
| Lactose | 1.56 | 0.85 | 0.77 |
| Microcrystalline | 1.56 | 0.81 | 0.83 |
| Cellulose | | | |
| Glucose | 1.60 | 0.81 | 0.83 |
| Magnesium Stearate | 1.16 | 0.85 | 0.89 |
| Croscarmellose Sodium | 1.47 | 0.80 | 0.89 |
| Starch | 1.49 | 0.86 | 0.90 |
| Colloidal Silicon | 1.47 | 0.85 | 0.89 |
| Dioxide | | | |
| Sodium Lauryl Sulfate | 1.44 | 0.82 | 0.90 |
| Sodium Starch | 1.43 | 0.72 | 0.90 |
| Glycolate | | | |
| Povidone | 1.41 | 1.02 | 1.10 |
| Mannitol | 1.49 | 0.87 | 0.91 |
| Talc | 1.42 | 0.82 | 0.92 |

### Formulation Development

Direct Compression Process

All materials were passed through a 710 micron sieve screen (25 mesh) prior to blending. The method for each batch involved addition of half the portion of lactose followed by Compound (Ia) 2HCl, microcrystalline cellulose, (sodium starch glycolate for formulation trials 3 and 4) and the remaining half portion of lactose. The materials were blended for approximately 5 minutes. To this blend the magnesium stearate was added and further blended for approximately 2 minutes. The tablets were compressed on a Manesty F3 single press with 8 mm round tooling. The pharmaceutical composition is as should in Table 7.

TABLE 7
Formulation for Compound (Ia) 2HCl 1 mg Tablets

| | Formulation 1 | Formulation 2 | Formulation 3 | Formulation 4 |
|---|---|---|---|---|
| | Amount/Tablet | Amount/Tablet | Amount/Tablet | Amount/Tablet |
| Material | (mg) | (mg) | (mg) | (mg) |
| (Ia) 2HCl | 1.0 | 1.0 | 1.0 | 1.0 |
| Lactose | 138.0 | 134.5 | 130.0 | 128.5 |
| Microcrystalline | 45.0 | 46.0 | 45.0 | 46.0 |
| Cellulose | | | | |
| Starch | 15.0 | 17.0 | 15.0 | 17.0 |
| Sodium | N/A | N/A | 8.0 | 6.0 |
| Starch | | | | |
| Glycolate | | | | |
| Magnesium | 1.0 | 1.5 | 1.0 | 1.5.0 |
| Stearate | | | | |
| Total | 200 mg | 200 mg | 200 mg | 200 mg |

An additional two formulations (based on Formulation 1 and Formulation 3 from Table 7) were prepared with a nominal weight of 200 mg per tablet. The API was ground in a mortar and pestle prior to screening and blending.

TABLE 8
Formulation for Compound (Ia) 2HCl 90 mg Tablets

| | Formulation F1 | Formulation F3 |
|---|---|---|
| Material | Amount/Tablet (mg) | Amount/Tablet (mg) |
| Compound (Ia) | 90.0 | 90.0 |
| Lactose | 49.0 | 41.0 |
| Microcrystalline | 45.0 | 45.0 |
| Cellulose | | |
| Starch | 15.0 | 15.0 |
| Sodium Starch | N/A | 8.0 |
| Glycolate | | |
| Magnesium Stearate | 1.0 | 1.0 |
| Total | 200 mg | 200 mg |

Assay was performed by HPLC in six random samples, the results are shown in Table 9:

TABLE 9
HPLC Assay Results

| | 1 mg | 1 mg | 1 mg | 1 mg | 90 mg | 90 mg |
|---|---|---|---|---|---|---|
| | Formulation | Formulation | Formulation | Formulation | Formulation | Formulation |
| Tablet | 1 (%) | 2 (%) | 3 (%) | 4 (%) | F1 (%) | F3 (%) |
| 1 | 99.5 | 105.2 | 89.4 | 129.0 | 95.6 | 101.7 |
| 2 | 146.5 | 83.8 | 93.2 | 102.2 | 98.1 | 100.3 |
| 3 | 107.3 | 139.4 | 125.3 | 89.3 | 97.9 | 84.1 |
| 4 | 86.7 | 90.4 | 96.8 | 86.3 | 89.4 | 102.5 |
| 5 | 95.0 | 95.5 | 130.5 | 88.7 | 98.3 | 84.4 |
| 6 | 95.8 | 105.1 | 90.2 | 116.2 | 89.6 | 101.4 |
| RSD (%) | 20.29 | 19.00 | 17.83 | 17.10 | 4.46 | 9.34 |

300 Mg Tablet

Two formulations were prepared for Comound (Ia) 2HCl 1 mg Tablets, with a nominal weight of 300 mg per tablet (see Table 10). Compound (Ia) 2HCl was ground in a mortar and pestle. All materials were passed through a 710 micron sieve screen prior to blending. The method for each batch involved addition of a third of the portion of lactose followed by the Compound (Ia) 2HCl. This was blended (using the “bag” blending technique) for approximately 3 minutes.

Separately, Aerosil (fumed silica) was sieved with a third of the portion of lactose. To the bag, the microcrystalline cellulose, Aerosil/lactose mixture, starch, (sodium starch glycolate for Formulation 6) and the remaining third portion of lactose were added and further blended for approximately 3 minutes. To this blend the magnesium stearate was added and further blended for approximately 1 minute. The tablets were compressed on a Manesty F3 single press with 10 mm round tooling.

TABLE 10
Formulation for Compound (Ia) 2HCl 1 mg Tablets

| | Formulation5 | Formulation 6 |
|---|---|---|
| Material | Amount/Tablet(mg) | Amount/Tablet (mg) |
| Compound (Ia) 2HCl | 1.0 | 1.0 |
| Lactose | 206.0 | 194.0 |
| Aerosil ® | 1.5 | 1.5 |
| Microcrystalline | 67.5 | 67.5 |
| Cellulose | | |
| Starch | 22.5 | 22.5 |
| Sodium Starch | N/A | 12.0 |
| Glycolate | | |
| Magnesium Stearate | 1.5 | 1.5 |
| Total | 300 mg | 300 mg |

Assay was performed by HPLC and the results are shown in Table 11.

TABLE 11
HPLC Assay Results for Formulation 5 and 6

| | 1 mg | 1 mg |
|---|---|---|
| Tablet | Formulation 5 (%) | Formulation 6 (%) |
| 1 | 99.0 | 85.1 |
| 2 | 99.2 | 90.9 |
| 3 | 101.5 | 85.5 |
| 4 | 101.9 | 85.1 |
| 5 | 101.1 | 87.4 |
| 6 | 98.5 | 86.7 |
| Average | 100.2 | 86.8 |
| RSD | 1.5 | 2.6 |

The formulations showed improved blend flow properties. The tablet appearance was improved and no mottling was observed. The content uniformity results for 6 tablets showed much more consistency. The physical attribute of hardness is more tightly controlled and friability fell within the acceptance criteria (see Table 12).

TABLE 12
Results for Formulation 5

| Test | Results |
|---|---|
| Assay by HPLC of 6 tablets | 92.2% |
| | 90.7% |
| | 89.6% |
| | 97.9% |
| | 89.8% |
| | 95.3% |
| | Average = 92.6% |
| | RSD = 9.3% |
| Individual weight of 20 tablets | Average = 308 mg |
| | RSD = 0.5% |
| Disintegration 6 tablets | 21 sec. |
| Friability | 0.2% |
| Hardness of 20 tablets | Min = 82 (N) |
| | Max = 88 (N) |
| | Average = 85 (N) |
| | RSD = 2.3% |

Blend Bulk and Tap Density

Measurement of the bulk and tap density provides information on the flow and compressibility of the blend. Bulk and tap density and the subsequent Carr's index were determined on the Formulation 5 blend and the results are given in Table 13.

TABLE 13
Bulk and Tap Density of Formulation 5

| | Bulk Density | Tap Density | Carr's Index |
|---|---|---|---|
| Sample | (g/mL) | (g/mL) | (%) |
| Blend-Formula 5 | 0.55 | 0.71 | 22.47 |

Colourant Formulation

A colourant was added to the formulation to ensure that the active tablets will be consistent in appearance (requirement for the clinical batches for future GMP manufacture). The formulation details are provided below in Table 14.

TABLE 14
Formulation for Compound (Ia) 2HCl 1 mg, 15 mg, 90 mg Tablets

| | Formulation 5 | Formulation 7 | Formulation 8 |
|---|---|---|---|
| | Amount/Tablet | Amount/Tablet | Amount/Tablet |
| Material | (mg) | (mg) | (mg) |
| Compound | 1.0 | 90.0 | 15.0 |
| I(a) | | | |
| Lactose | 201.2 | 117.0 | 189.0 |
| Microcrystalline | 67.5 | 67.5 | 67.5 |
| Cellulose | | | |
| Aerosil ® | 1.5 | 1.5 | 1.5 |
| Starch | 22.5 | 22.5 | 22.5 |
| Magnesium | 1.5 | 1.5 | 1.5 |
| Stearate | | | |
| Yellow 10 | 4.8 | N/A | 3.0 |
| Iron Oxide | | | |
| Total | 300 mg | 300 mg | 300 mg |

The analysis was performed according TM1373 and the results are summarised in Table 15.

TABLE 15
Results for Formulation for Compound (Ia) 2HCl 1 mg, 15 mg, 90 mg Tablets

| | 1 mg Tablets | 15 mg Tablets | 90 mg Tablets |
|---|---|---|---|
| Test | (Formulation 5) | (Formulation 8) | (Formulation 7) |
| Appearance | Light yellow round | Light yellow round | Light yellow round |
| | tablet | tablet | tablet |
| Assay by HPLC of | 98.8% | 98.4% | 100.1% |
| 6 tablets | 105.3% | 96.4% | 100.5% |
| | 100.4% | 90.3% | 98.3% |
| | 98.7% | 95.8% | 98.3% |
| | 94.1% | 97.8% | 100.7% |
| | 100.8% | 95.9% | 99.4% |
| | Average = 99.7% | Average = 95.8% | Average = 99.6% |
| | RSD = 3.7% | RSD = 3.0% | RSD = 1.07% |
| Individual weight of 10 | Average = 306 mg | Average = 305 mg | Average = 300 mg |
| tablets | RSD = 0.7% | RSD = 0.9% | RSD = 1.7% |
| Disintegration of 6 | 26 sec. | 2 min 03 sec | 6 min 20 sec |
| tablets | | | |
| Friability | 0.2% | 0.40% | 0.67% |
| Hardness of 10 tablets | Min = 85(N) | Min = 77(N) | Min = 45(N) |
| | Max = 94(N) | Max = 87(N) | Max = 54(N) |
| | Average = 90(N) | Average = 81(N) | Average = 50(N) |
| | RSD = 4.0% | RSD = 3.6% | RSD = 7.1% |
| Thickness (mm) of 10 | Min = 3.50 mm | Min = 3.48 mm | Min = 3.65 mm |
| tablets | Max = 3.54 mm | Max = 3.55 mm | Max = 3.70 mm |
| | Average = 3.52 mm | Average = 3.52 mm | Average = 3.68 mm |
| | RSD = 0.3% | RSD = 0.7% | RSD = 0.3% |

GLP Tablet Manufacture

Prior to manufacture of the GMP clinical batches, GLP batches would be produced in order to run the manufacturing process at the intended scale and generate stability data by performing an indicative stability study.

As discovered above, in order to improve the homogeneity of the blend and the appearance of the tablets, the API required milling prior to GLP tablet manufacture.

Particle size analysis was performed on the API and the results are presented in Table 16.

TABLE 16
Particle Size Distribution of Compound (Ia) 2HCl
Particle Size (μm)

| D (0.1) | D (0.5) | D (0.9) |
|---|---|---|
| 1.2 | 38.6 | 133.3 |

The analytical results obtained for the three GLP Batches are presented in Table 17.

TABLE 17
Results for GLP Batches

| Test | 1 mg Tablets | 15 mg Tablets | 90 mg Tablets |
|---|---|---|---|
| Appearance | Pale yellow, | Pale yellow, | Pale yellow, |
| | round, | round, | round, |
| | compressed | compressed | compressed |
| | tablet. | tablet. | tablet. |
| Assay (HPLC) | 101.8% | 97.8% | 98.6% |
| Content Uniformity | Ave: 103% | Ave: 97% | Ave: 97% |
| (HPLC) | RSD: 1.6% | RSD: 5.0% | RSD: 1.5% |
| pH | 5.83 | 3.27 | 2.47 |
| Dissolution | Ave: 90% | Ave: 101% | Ave: 106% |
| (HPLC) | Range: 84-93% | Range: 90-117% | Range: 95-111% |
| Hardness of 30 | Ave: 76.3 N | Ave: 62.6 N | Ave: 67.7 N |
| tablets | | | |
| Thickness (mm) | Ave: 3.45 mm | Ave: 3.80 mm | Ave: 3.49 mm |
| of 30 tablets | | | |

Dissolution Studies of Formulations

Medium Selection

Selection of the dissolution medium is based on the solubility data and the dose range in order to ensure that sink conditions are met. The term ‘sink conditions’ is defined as the volume of medium at least greater than three times that required to form a saturated solution of drug substance.

Sink Conditions:

The sink conditions test was performed as per USP 38 <1092> in the following dissolution medium:
1. HCl 0.01 N,
2. Buffer pH 6.8,
3. Water.

Solubility determinations were performed on 75 mg of Compound (Ia) HCl dissolved in 250 mL of different media at 37° C. and stirred slightly. The λₘₐₓ was determined by UV scan between 200-400 nm. Refer to Table 18 for the results.

TABLE 18
Solubility and λₘₐₓ of Compound (Ia) 2HCl

| | Time to | | | Mean Absorbance |
|---|---|---|---|---|
| Medium | Dissolve | Appearance | λₘₐₓ | at λₘₐₓ |
| 0.01N | 1 min 22 sec | Clear, | 254.5 | 0.103 |
| Hydrochloric | | homogeneous, | | |
| Acid (pH 2.0) | | translucent to | | |
| | | light yellow. | | |
| Phosphate | 1 min 02 sec | White, turbid, | 256.5 | 0.085 |
| Buffer (pH 6.8) | | heterogeneous | | |
| | | liquid. | | |
| Purified Water | 21 sec | Clear, | 256 | 0.058 |
| | | homogeneous, | | |
| | | translucent to | | |
| | | light yellow. | | |

Determination of Intrinsic Dissolution Rate (IDR)

The IDR is determined by monitoring the drug release rate of a compressed disc of the pre-formulated drug. The IDR is independent of formulation effects and measures the intrinsic properties of the drug and salts as a function of dissolution media effects such as pH and ionic strength. A comparison of the IDR of the drug in water with that obtained in acid and alkali will provide a measure of the drug's ability to control its immediate microenvironment.

Procedure

The IDR was determined by preparing a compressed disc containing 200 mg of Compound (Ia) 2HCl using slow compression. All metal surfaces were pre-lubricated using 4 drops of a 5% w/v solution of stearic acid in chloroform. The solvent was allowed to evaporate, the Compound (Ia) 2HCl added and the sample slowly compressed to 6 tonne. This pressure was maintained for 4 minutes to ensure adequate compression.

The discs were then rotated at 100 rpm, 25 mm from the bottom of a 1 litre flat bottomed dissolution vessel containing 1 litre of fluid maintained at 37° C. Manual sampling, with a sampling volume of 5 mL, was employed at 5, 10, 15, 30 and 60 minutes. The amount of drug released was then monitored, by HPLC. Compound (Ia) 2HCl was evaluated in 0.01N HCl (equivalent to gastric pH), phosphate buffer pH 6.8 (intestinal) and distilled water. Refer Table 19 for the percentage of Compound (Ia) 2HCl released.

TABLE 19
Intrinsic Dissolution Rate of Compound (Ia) 2HCl

| | % Compound (Ia) HCl Released | | | | | |
|---|---|---|---|---|---|---|
| | t = 0 | t = 5 | t = 10 | t = 15 | t = 30 | t = 60 |
| Sample | mins | mins | mins | mins | mins | mins |
| Water (1) | 0 | 84 | 101 | 101 | 100 | 100 |
| Water (2) | 0 | 71 | 98 | 100 | 102 | 100 |
| 0.01N 2HCl (1) | 0 | 70 | 97 | 98 | 99 | 98 |
| 0.01N 2HCl (2) | 0 | 80 | 100 | 101 | 102 | 101 |
| Phosphate buffer | 0 | 68 | 85 | 72 | 73 | 59 |
| pH 6.8 (1) | | | | | | |
| Phosphate buffer | 0 | 60 | 81 | 83 | 77 | 60 |
| pH 6.8 (2) | | | | | | |

Dissolution Method

For immediate release dosage forms, the duration of the dissolution procedure is typically 30 to 60 minutes with a single time point test. Products showing less than ideal solubilities (<10 mg/mL) typically demonstrate release profiles showing a gradual increase reaching between 85% and 100% at around 30 to 45 minutes. Thus, dissolution time points in the range from 15, 30, 45 and 60 minutes are common for immediate release products. Consequently, sampling points of 5, 10, 15, 20, 30, 45 and 60 minutes were employed throughout this study.

Reagents and Equipment

Deionised (DI) water

Concentrated hydrochloric acid (HCl) 37% AR grade or equivalent

Ethanol, ACS grade

Compound (Ia) HCl reference standard

Calibrated Dissolution System (with paddles)

Calibrated analytical balance

Calibrated Thermometer

Disposable 0.45 μm GHP filters

Disposable 10 mL syringes

Stirrer/Hot plate

Ultra Sonic bath

Vacuum apparatus and filter

Medium Preparation (0.1 N HCl)

Mix 8.5 mL of concentrated 2HCl with 1000 mL of DI water. Set some aside to prepare standards. Filter the remaining through 0.45 μm filter under vacuum to degas. This preparation may be scaled up or down as appropriate.

Mobile Phase Preparation

5 mM Ammonium Hydroxide

Dilute 1.0 mL of 28% ammonia solution to 25 mL with deionised water. Further dilute this solution 2.0 mL to 250 mL with water. This preparation may be scaled up or down as appropriate.

5 mM Ammonium Acetate, pH 8.2 solution

Dissolve approximately 0.69 g of ammonium acetate in 1800 mL water. Add 150 mL 5 mM ammonium hydroxide. Check pH and, if necessary, adjust with 5 mM ammonium hydroxide to a pH of 8.20±0.10. This preparation may be scaled up or down as appropriate.

Mobile Phase

To 300 mL of 5 mM Ammonium Acetate, pH 8.2 solution add 700 mL of acetonitrile. Filter through a 0.45 μm membrane filter and degas. This preparation may be scaled up or down as appropriate.

Standard Preparation

Prepare standards in duplicate (label as standards 1 and 2).

Stock Standard

Weigh accurately 20 mg of Compound (Ia) 2HCl reference standard in duplicate and transfer into individual 200 mL volumetric flasks. Add ethanol to about half of the volumetric flask and sonicate for 5 minutes to dissolve the solids. Dilute to volume with ethanol and mix well. Determine the water content of the standard by Karl Fischer at the time of use, using 0.1 g of standard in duplicate.

Working Standard

Dilute 2.0 mL of the stock standard to 100.0 mL with medium and mix well. (2 μg/mL of Compound (Ia) 2HCl)

Dissolution Procedure

Dissolution Parameters

Apparatus: USP II (Paddle)

Medium: 0.1 N HCl

Speed: 50 rpm

Temperature: 37.0±0.5° C.

Sampling Time: 30 minutes

Sampling Volume: 15 mL

Note: Volume for 1 mg tablet: 500 mL

Volume for 15 mg and 90 mg tablet: 900 mL

Dissolution of Sample

Assemble the dissolution baths, add medium and allow to equilibrate to temperature. Record the actual temperature of the medium for each vessel. Weigh six tablets individually and record the weights. Set the apparatus in position and start rotating the paddles at the specified speed. Drop each tablet into an individual vessel and start the time measurement. Make sure there is sufficient time allowed between each tablet addition to ensure adequate sampling time between each vessel.

At 30 minutes, withdraw a 15 mL aliquot from each vessel. Then filter using 0.45 μm GHP filter. Discard the first 4 mL of filtrate.

For 1 mg strength; proceed to HPLC analysis. For 15 mg strength, dilute 3.0 mL to 25.0 mL with medium and mix well prior to HPLC analysis. For 90 mg strength, dilute 2.0 mL to 100.0 mL with medium and mix well prior to HPLC analysis.

The dilution step can be adjusted to suit the available glassware as long as the final concentration is maintained.

Chromatographic System

Chromatographic Parameters

Column: Waters XBridge BEH C18 column, 3.5 μm, 4.6×150 mm

Column Temp: 40° C.

Mobile Phase: 30% 5 mM Ammonium Acetate, pH 8.2, 70% Acetonitrile

Flow Rate: 1.0 mL/min

Run Time: 8 minutes

Injection Volume: 20 μL

Detection: UV at 260 nm

Sample Temperature: Ambient (25° C.)

Needle Wash: 70% Acetonitrile

System Suitability

Perform duplicate injections of the blank (diluent) solution. Ensure that there is no significant interference at the retention times of the active.

Perform six replicate injections of the first standard solution. Compound I(a) 2HCl elutes at approximately 4 minutes. Calculate the relative standard deviation of the peak areas and retention times of the Compound (Ia) 2HCl peak.

Perform duplicate injections of the second standard solution. Calculate the agreement between standards as follows:

$$\% Agreement = \frac{{As}_{1} \times {Ws}_{2}}{{Ws}_{1} \times {As}_{2}} \times 100$$

where:
As1=mean area of Compound (Ia) 2HCl peak in Standard 1
Ws1=weight of Compound (Ia) 2HCl in Standard 1 (mg)
As2=mean area of Compound (Ia) 2HCl peak in Standard 2
Ws2=weight of Compound (Ia) 2HCl in Standard 2 (mg)

The system suitability limits in Table 20 must be met for each analytical run.

TABLE 20
System Suitability Parameters

| System Suitability Parameter | Acceptance Criterion |
|---|---|
| Blank | No significant interference at |
| | RT of peaks of interest |
| Tailing Factor | 0.8-2.0 |
| % RSD of 6 Standard | Not More Than 2.0% |
| Injections-Peak Area | |
| % RSD of 6 Standard | Not More Than 1.0% |
| Injections-Retention Time | |
| Agreement between Standard | 98.0-102.0% |
| 1 and 2 preparations | |
| Drift between Bracketing Standards | Not More Than 2.0% |

Calculations

Calculate the Compound (Ia) 2HCl dissolved during the test according to the following equation:

$$\% Dissolved = \frac{P \times Ws \times ( \frac{( 100 - \% Std water )}{100} )}{As \times Ds \times 100} \times \frac{Au \times Du}{Wu}$$

where:
Ws=Weight of Compound (Ia) 2HCl in standard (mg)
P=Potency of Compound (Ia) 2HCl standard (% anhydrous)
As=Mean bracketed area of Compound (Ia) 2HCl peak in standard
Ds=Dilution factor of standard (10000)
Au=Area of Compound (Ia) 2HCl peak in sample
Du=Dilution factor of sample (500 for 1 mg, 7500 for 15 mg, 45000 for 90 mg)
Wu=Label Claim of Tablet (mg)

Interpretation

The requirements are met if the quantities of active ingredient dissolved from the dosage units tested conform to Table 2. Continue testing through the three stages unless the results conform at either S1 or S2. The quantity, Q, is the specification for the amount of dissolved active ingredient expressed as a percentage of the labelled content of the dosage unit; the 5%, 15%, and 25% values in Table 21 are percentages of the labelled content so that these values and Q are in the same terms. Refer to the specification sheet for the ‘Q’ value.

TABLE 21
Dissolution Criteria

| | Number | |
|---|---|---|
| Stage | Tested | Acceptance Criteria |
| S1 | 6 | Each unit is not less than Q + 5%. |
| S2 | 6 | Average of 12 units (S1 + S2) is equal to or greater |
| | | than Q, and no unit is less than Q − 15%. |
| S3 | 12 | Average of 24 units (S1 + S2 + S3) is equal to or |
| | | greater than Q, not more than 2 units are less than |
| | | Q − 15%, and no unit is less than Q − 25%. |

C_{std} = Concentration of standard (mg/mL)

Evaluation of Dissolution Parameters for Compound (Ia) 2HCl Tablets.

For immediate release products, the paddle method (Apparatus 2) is routinely used for tablet formulations at an agitation speed of 50 to 75 rpm. In order to determine the optimal dissolution parameters for Compound (Ia) 2HCl Tablets, the following configurations were investigated for Compound (Ia) 2HCl Tablets, 1 mg and the resulting dissolution profiles compared. Purified water was used as the dissolution medium at a temperature of 37° C.±0.5° C. for each study. Refer to Tables 22-24 for the dissolution results obtained at varying agitation speeds.

TABLE 22
Dissolution of Compound (Ia) 2HCl Tablets, 1 mg at
50 rpm in Water

| | % Released | | | | | |
|---|---|---|---|---|---|---|
| Dissolution | Vessel | Vessel | Vessel | Vessel | Vessel | Vessel |
| Time (min) | #1 | #2 | #3 | #4 | #5 | #6 |
| 0 | 0 | 0 | 0 | 0 | 0 | 0 |
| 5 | 0 | 0 | 0 | 0 | 0 | 0 |
| 15 | 27 | 40 | 39 | 30 | 42 | 40 |
| 30 | 43 | 53 | 48 | 51 | 54 | 58 |
| 45 | 57 | 59 | 51 | 60 | 59 | 65 |
| 60 | 58 | 59 | 58 | 62 | 66 | 61 |

TABLE 23
Dissolution of Compound (Ia) 2HCl Tablets, 1 mg at 75 rpm in Water

| | % Released | | | | | |
|---|---|---|---|---|---|---|
| Dissolution | Vessel | Vessel | Vessel | Vessel | Vessel | Vessel |
| Time (min) | #1 | #2 | #3 | #4 | #5 | #6 |
| 0 | 0 | 0 | 0 | 0 | 0 | 0 |
| 5 | 0 | 1 | 6 | 3 | 0 | 11 |
| 10 | 48 | 49 | 51 | 45 | 41 | 53 |
| 15 | 55 | 57 | 62 | 53 | 60 | 64 |
| 30 | 62 | 63 | 65 | 69 | 61 | 66 |
| 45 | 70 | 69 | 69 | 68 | 69 | 71 |
| 60 | 74 | 74 | 72 | 77 | 76 | 70 |

TABLE 24
Dissolution of Compound (Ia) 2HCl Tablets, 1 mg at 100 rpm in Water

| | % Released | | | | | |
|---|---|---|---|---|---|---|
| Dissolution | Vessel | Vessel | Vessel | Vessel | Vessel | Vessel |
| Time (min) | #1 | #2 | #3 | #4 | #5 | #6 |
| 0 | 0 | 0 | 0 | 0 | 0 | 0 |
| 5 | 5 | 0 | 0 | 1 | 4 | 17 |
| 10 | 48 | 39 | 35 | 47 | 48 | 53 |
| 15 | 52 | 51 | 48 | 58 | 58 | 62 |
| 30 | 61 | 63 | 58 | 66 | 59 | 67 |
| 45 | 59 | 69 | 61 | 67 | 69 | 72 |
| 60 | 65 | 72 | 71 | 68 | 69 | 75 |

Water was used as dissolution medium in the three testes, as recommended by USP. After 60 minutes at the highest speed, the drug percentage released was below 75%.

Another profile was performed with Compound (Ia) 2HCl Tablets, 1 mg using 0.1 N HCl as dissolution medium at a temperature of 37° C.±0.5° C. An agitation speed of 75 rpm was selected. The HCl 0.1 M dissolution media results are presented in Table 25.

TABLE 25
Dissolution of Compound (Ia) 2HCl Tablets, 1 mg at 75 rpm in
0.1N HCl

| | % Released | | | | | |
|---|---|---|---|---|---|---|
| Dissolution | Vessel | Vessel | Vessel | Vessel | Vessel | Vessel |
| Time (min) | #1 | #2 | #3 | #4 | #5 | #6 |
| 0 | 0 | 0 | 0 | 0 | 0 | 0 |
| 5 | N.A. | 94 | 93 | 99 | 99 | 97 |
| 10 | 103 | 97 | 94 | 101 | 101 | 100 |
| 15 | 101 | 95 | 93 | 100 | 100 | 99 |
| 30 | 99 | 93 | 92 | 98 | 99 | 97 |
| 45 | 99 | 93 | 91 | 97 | 97 | 96 |
| 60 | 99 | 92 | 92 | 99 | 98 | 96 |

The results showed rapid dissolution which was around 100% of release in approximately 10 minutes. To determine the specific concentration of HCl, a comparison between 0.1 N HCl and 0.01 N HCl was performed for all tablet strengths and the results are presented in Table 26. Using USP (II) (Paddle), a medium of 0. 1 N HCl, speed of 50 rpm, temperature of 37.0±0.5° C., sampling time of 30 minutes, sampling volume of 15 mL, the dissolution profiles demonstrate that, using paddles at 50 rpm, >90% dissolution was achieved within 5 minutes for Compound (Ia) 2HCl Tablets, 1 mg and 15 mg and 15 minutes for EU-C-001 HCl Tablets, 90 mg.

TABLE 26
Dissolution of Compound (Ia) 2HCl Tablets at 50 rpm in
0.1N and 0.01N HCl

| | % Released | | | | | |
|---|---|---|---|---|---|---|
| | Vessel | Vessel | Vessel | Vessel | Vessel | Vessel |
| | #1 | #2 | #3 | #4 | #5 | #6 |
| | 1 mg | 1 mg | 15 mg | 15 mg | 90 mg | 90 mg |
| Dissolution | 0.1N | 0.01N | 0.1N | 0.01N | 0.1N | 0.01N |
| Time (min) | HCl | HCl | HCl | HCl | HCl | HCl |
| 0 | 0 | 0 | 0 | 0 | 0 | 0 |
| 5 | 96 | 90 | 96 | 97 | 40 | 39 |
| 10 | 101 | 95 | 106 | 101 | 76 | 75 |
| 15 | 103 | 96 | 103 | 103 | 109 | 98 |
| 20 | 103 | 96 | 103 | 98 | 116 | 99 |
| 30 | 104 | 96 | 109 | 99 | 121 | 97 |
| 45 | 104 | 96 | 105 | 102 | 114 | 100 |
| 60 | 106 | 97 | 108 | 106 | 120 | 104 |

The profile showed no significant difference in the percentage released between 0.1 N HCl and 0.01 N HCl for all tablet strengths. Overall, it was observed that optimum release was obtained using paddles with an agitation speed of 50 rpm, in a 0.1 N HCl dissolution medium, with a dissolution rate of approximately 100% for the Compound (Ia) HCl Tablets, 1 mg, 15 mg and 90 mg being achieved within 30 minutes under these conditions.

1. A pharmaceutical composition in the form of a tablet comprising:
(i) a compound of Formula (I) or a pharmaceutically acceptable salt, solvate or prodrug thereof: 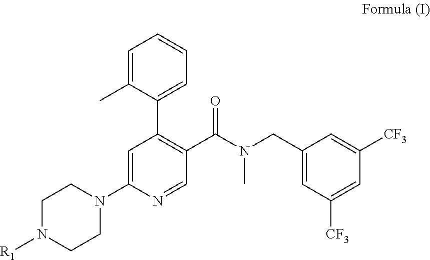
wherein R₁ is H or C₁₋₄ alkyl; and
wherein the compound of Formula (I) or the pharmaceutically acceptable salt, solvate or prodrug thereof within the composition has a D(0.5) particle size distribution of less than about 60 μm;
(ii) at least one diluent selected from the group consisting of lactose, sorbitol, dibasic calcium phosphate dihydrate, calcium sulphate dihydrate, calcium carbonate, croscarmellose sodium, calcium phosphate, calcium hydrogen phosphate dihydrate, crospovidone, ferric oxide, magnesium carbonate, magnesium oxide, sucrose, or sodium chloride, wherein the at least one diluent is present in the composition in an amount from about 35% to about 70% wt/wt based on the total weight of the composition;
(iii) at least one lubricant selected from the group consisting of magnesium stearate, stearic acid, calcium stearate, paraffin, sodium lauryl sulphate, sodium benzoate, castor oil hydrogenated, glyceryl monostearate, glyceryl behenate, sodium stearyl fumarate, mineral oil, polaxamer, PEG 400, PEG 600, or PEG 8000, wherein the at least one lubricant is present in the composition in an amount from about 0.1% to about 2% wt/wt based on the total weight of the composition;
(iv) at least one disintegrant selected from the group consisting of microcrystalline cellulose, alginic acid, citric acid, croscamellose sodium, carboxy methyl cellulose calcium, cysteine HCl, methyl cellulose, polyoxy stearate, sodium starch glycolate, sodium alginate, or carboxy methyl cellulose sodium, wherein the at least one disintegrant is present in the composition in an amount from about 20% to about 30% wt/wt based on the total weight of the composition;
(v) at least one binder selected from the group consisting of starch, gelatin, glucose, polyvinyl pyrrolidone (Povidone), carboxymethylcellulose, acacia, candelilla wax, carnuba wax, cornstarch, glyceryl behenate, hypromellose, or polyethylene oxide, wherein the at least one binder is present in the composition in an amount from about 5% to about 15% wt/wt based on the total weight of the composition; and
(vi) at least one anti-caking agent selected from the group consisting of fumed silica, silicon dioxide, or talc, wherein the at least one anti-caking agent is present in the composition in an amount from about 0.2% to about 2% wt/wt based on the total weight of the composition.

2. The pharmaceutical composition according to claim 1, wherein R₁ is selected from H, methyl, ethyl, n-propyl or iso-propyl.

3. The pharmaceutical composition according to claim 1, wherein at least one diluent is selected from the group consisting of lactose, sorbitol, or sucrose.

4. The pharmaceutical composition according to claim 1, wherein at least one lubricant is selected from the group consisting of magnesium stearate, stearic acid, calcium stearate, PEG 400, PEG 600, or PEG 8000.

5. The pharmaceutical composition according to claim 1, wherein at least one disintegrant is selected from the group consisting of microcrystalline cellulose, sodium starch glycolate, carboxy methyl cellulose calcium, methyl cellulose, or carboxy methyl cellulose sodium.

6. The pharmaceutical composition according to claim 1, wherein at least one binder is selected from the group consisting of starch, gelatin, glucose, acacia, candelilla wax, carnuba wax, or cornstarch.

7. The pharmaceutical composition according to claim 1, wherein the anti-caking agent is fumed silica.

8. The pharmaceutical composition according to claim 1, wherein the diluent is lactose.

9. The pharmaceutical composition according to claim 1, wherein the lubricant is magnesium stearate.

10. The pharmaceutical composition according to claim 1, wherein the disintegrant is microcrystalline cellulose.

11. The pharmaceutical composition according to claim 1, wherein the binder is starch.

12. The pharmaceutical composition according to claim 1, wherein the compound of Formula (I) or the pharmaceutically acceptable salt, solvate or prodrug thereof is present in the composition in an amount from about 0.1% to about 50% wt/wt based on the total weight of the composition.

13. The pharmaceutical composition according to claim 1, further comprising at least one coating selected from the group consisting of hydroxypropyl cellulose, hydroxypropyl methylcellulose, hydroxypropyl methyl cellulose phthalate, methyl cellulose, methacrylic acid copolymer, erthrosine sodium, or sodium propionate.

14. (canceled)

15. The pharmaceutical composition according to claim 1, wherein the compound of Formula (I) is:

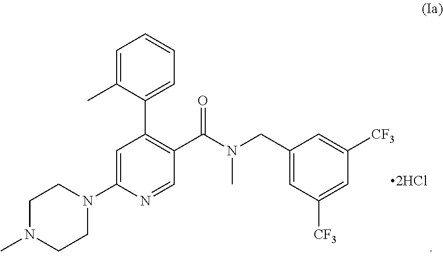 [SMILES: Cc1ccccc1-c1cc(N2CCN(C)CC2)ncc1C(=O)N(C)Cc1cc(C(F)(F)F)cc(C(F)(F)F)c1]

16. A method for treating over-expression of hyper-phosphorylated tau protein (τ) in the brain, post-concussion syndrome (PCS), chronic traumatic encephalopathy (CTE) or elevated intracranial pressure in a subject in need thereof, the method comprising administering to the subject a pharmaceutical composition in the form of a tablet comprising:
(i) a compound of Formula (I) or a pharmaceutically acceptable salt, solvate or prodrug thereof: 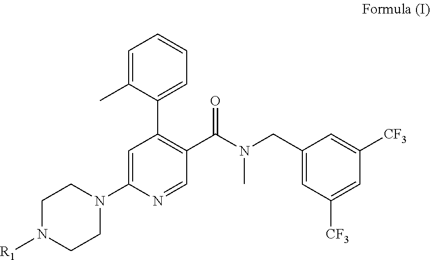
wherein R₁ is H or C₁₋₄ alkyl; and
wherein the compound of Formula (I) or the pharmaceutically acceptable salt, solvate or prodrug thereof within the composition has a D(0.5) particle size distribution of less than about 60 μm;
(ii) at least one diluent selected from the group consisting of lactose, sorbitol, dibasic calcium phosphate dihydrate, calcium sulphate dihydrate, calcium carbonate, croscarmellose sodium, calcium phosphate, calcium hydrogen phosphate dihydrate, crospovidone, ferric oxide, magnesium carbonate, magnesium oxide, sucrose, or sodium chloride, wherein the at least one diluent is present in the composition in an amount from about 35% to about 70% wt/wt based on the total weight of the composition;
(iii) at least one lubricant selected from the group consisting of magnesium stearate, stearic acid, calcium stearate, paraffin, sodium lauryl sulphate, sodium benzoate, castor oil hydrogenated, glyceryl monostearate, glyceryl behenate, sodium stearyl fumarate, mineral oil, polaxamer, PEG 400, PEG 600, or PEG 8000, wherein the at least one lubricant is present in the composition in an amount from about 0.1% to about 2% wt/wt based on the total weight of the composition;
(iv) at least one disintegrant selected from the group consisting of microcrystalline cellulose, alginic acid, citric acid, croscamellose sodium, carboxy methyl cellulose calcium, cysteine HCl, methyl cellulose, polyoxy stearate, sodium starch glycolate, sodium alginate, or carboxy methyl cellulose sodium, wherein the at least one disintegrant is present in the composition in an amount from about 20% to about 30% wt/wt based on the total weight of the composition;
(v) at least one binder selected from the group consisting of starch, gelatin, glucose, polyvinyl pyrrolidone (Povidone), carboxymethylcellulose, acacia, candelilla wax, carnuba wax, cornstarch, glyceryl behenate, hypromellose, or polyethylene oxide, wherein the at least one binder is present in the composition in an amount from about 5% to about 15% wt/wt based on the total weight of the composition; and
(vi) at least one anti-caking agent selected from the group consisting of fumed silica, silicon dioxide, or talc, wherein the at least one anti-caking agent is present in the composition in an amount from about 0.2% to about 2% wt/wt based on the total weight of the composition.

17. (canceled)

18. The method according to claim 16, wherein the method for treating elevated intracranial pressure is a method for treating traumatic brain injury.

19. The method according to claim 16, wherein the method for treating elevated intracranial pressure is a method for treating stroke.

20. The method according to claim 16, wherein the method for treating elevated intracranial pressure is a method for treating PCS.
